(19) United States
(12) Patent Application Publication
(10) Pub. No.: US 2021/0153838 A1
(43) Pub. Date: May 27, 2021

(54) Method and Apparatus of Intelligent Analysis for Liver Tumor

(71) Applicants: Hsiao-Ching Nien, Taipei City (TW); YUAN High-Tech Development Co., Ltd., Taipei City (TW)

(72) Inventors: Hsiao-Ching Nien, Taipei City (TW); Ta-Hsiang Lin, New Taipei City (TW); Pei-Lien Chou, Taipei City (TW)

(21) Appl. No.: 16/952,238

(22) Filed: Nov. 19, 2020

Related U.S. Application Data
(63) Continuation-in-part of application No. 16/793,772, filed on Feb. 18, 2020, now abandoned.

(30) Foreign Application Priority Data
Nov. 21, 2019 (TW) 108142298

(51) Int. Cl. A61B 8/08; A61B 8/14; A61B 8/00; G16H 50/30; G06N 20/00

(52) U.S. Cl. CPC A61B 8/085; A61B 8/14; G06N 20/00; A61B 8/5207; G16H 50/30; A61B 8/4245

(57) ABSTRACT

A method of Intelligent Analysis is provided for liver tumor. It is a method using scanning acoustic tomography (SAT) with a deep learning algorithm for determining the risk of malignance for liver tumor. The method uses the abundant experiences of abdominal ultrasound specialists as a base to mark pixel areas of liver tumors in ultrasound images. The parameters and coefficients of empirical data are trained with the deep learning algorithm to establish a categorizer model reaching an accuracy rate up to 86 percent. Thus, with an SAT image, a help to doctor or ultrasound technician is obtained to determine the risk of malignance for liver tumor through the method, and to further provide a reference base for diagnosing liver tumor category.

### TECHNICAL FIELD OF THE INVENTION

The present invention relates to a method and apparatus for analyzing a liver tumor; more particularly, to coordinating scanning acoustic tomography (SAT) with a deep learning algorithm to determine the risk probability of malignant liver tumor, where, with a SAT image, a help to a doctor or ultrasound technician is immediately obtained for determining the risk probability of malignance of a liver tumor and a base of reference is further provided for diagnosing the liver tumor category.

### DESCRIPTION OF THE RELATED ARTS

Liver cancer is the fourth worldwide death cause. The most common causes of liver cancer in Asia are B-type and C-type hepatitis viruses and aflatoxin. The C-type hepatitis virus is a common cause in the United States and Europe. The liver cancers caused by steatohepatitis, diabetes, and triglyceride have become increasingly serious.

Surgery is currently the most direct method for treating liver cancers. However, early liver cancer diagnoses and postoperative patient-related prognostic indicators are also very important. A patient having a liver cancer confirmed by early diagnosis usually have more treatment options, where the treatment efficacy is shown by an improved survival rate of patients. Therefore, regular inspection and early diagnosis and treatment are the keys to improve the quality of life and to prolong the survival rate of patients.

In addition to early diagnoses including liver function blood test, B-type and C-type hepatitis virus, and A-type fetoprotein, abdominal ultrasound is an important test for liver disease, as studies indicated. An early study denoted that the liver blood tests of ⅓ patients with small HCC remained normal indexes for A-type fetoprotein. Ultrasound examination must be complemented for early detection of liver cancer. Furthermore, abdominal ultrasound examination has the features of quickness, easiness and non-radiation, which becomes an important tool for screening liver cancer.

The diagnosis of liver cancer is different from those of other cancers. Its confirmation does not require biopsy, but is directly obtained through imaging diagnosis like abdominal ultrasound, computed tomography (CT), and magnetic resonance imaging (MRI), etc. Its sensitivity and specificity are 0.78˜0.73 and 0.89˜0.93, 0.84˜0.83 and 0.99˜0.91, and 0.83 and 0.88, respectively.

SAT is convenient, but has its own limit. For example, operator experience, patient obesity, existence of liver fibrosis or cirrhosis, etc. would affect the accuracy of ultrasound. Therefore, when malignancy is detected out through SAT, a second imaging detection would be arranged, like CT or assisted diagnosis of MRI. Yet, these two detections have expensive costs for health care and lengthy examination schedules; and CT has consideration on more radiation exposure.

Hence, the prior arts do not fulfil all users' requests on actual use.

### SUMMARY OF THE INVENTION

The main purpose of the present invention is to coordinate SAT with a deep learning algorithm to determine the nature of a liver tumor, where the method of the invention is able to achieve an accuracy rate reaching as high as 86%, similar to that of CT or MRI, and thus provides physicians with radiation-free and safe SAT to rapidly and accurately diagnose liver tumor categories.

To achieve the above purpose, the present invention is a method of intelligent analysis (IA) for liver tumor, comprising steps of: (a) first step: providing a device of scanning acoustic tomography (SAT) to scan an area of liver of an examinee from an external position to obtain an ultrasonic image of a target liver tumor of the examinee; (b) second step: obtaining a plurality of existing ultrasonic reference images of benignant and malignant liver tumors; (c) third step: obtaining a plurality of liver tumor categories from the existing ultrasonic reference images based on the shading and shadowing areas of the existing ultrasonic reference images to mark a plurality of tumor pixel areas in the existing ultrasonic reference images and identify the liver tumor categories of the tumor pixel areas; (d) fourth step: obtaining the tumor pixel areas in the ultrasonic reference images to train a categorizer model with the coordination of a deep learning algorithm; and (e) fifth step: processing an analysis of the ultrasonic image of the target liver tumor of the examinee with the categorizer model to provide the analysis to a clinician to determine the target liver tumor a liver tumor category and predict a risk probability of malignance of the target liver tumor.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention; and

FIG. 2 is the block view.

### DESCRIPTION OF THE PREFERRED EMBODIMENT

The following description of the preferred embodiment is provided to understand the features and the structures of the present invention.

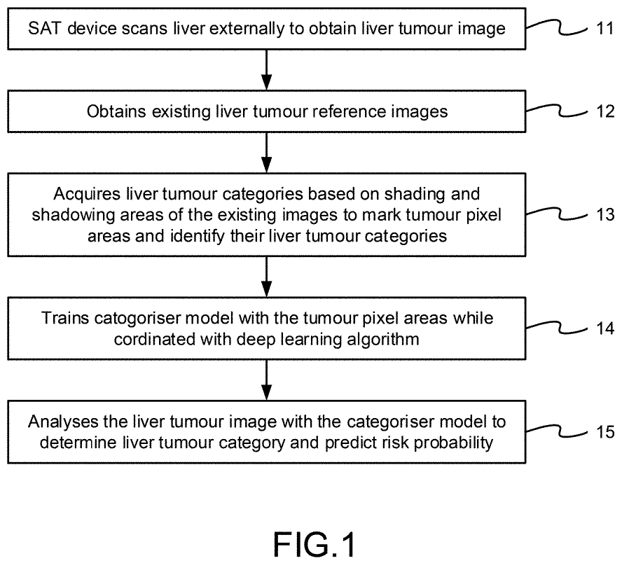

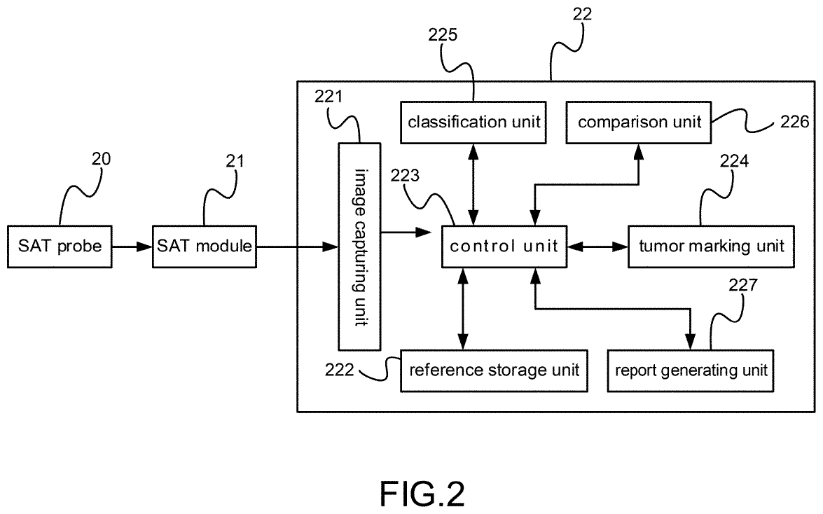

Please refer to FIG. 1 and FIG. 2, which are a flow view and a block view showing a preferred embodiment according to the present invention. As shown in the figures, the present invention is a method of intelligent analysis (IA) for liver tumor, comprising the following steps:

(a) First step 11: A device of scanning acoustic tomography (SAT) is provided to scan an area of liver of an examinee from an external position to obtain an ultrasonic image of a target liver tumor of the examinee.

(b) Second step 12: A plurality of existing ultrasonic reference images of benignant and malignant liver tumors are obtained.

(c) Third step 13: Based on the shading and shadowing areas of the existing ultrasonic reference images, a plurality of liver tumor categories of the existing ultrasonic reference images are acquired to mark a plurality of tumor pixel areas in the existing ultrasonic reference images and identify the liver tumor categories of the tumor pixel areas.

(d) Fourth step 14: The tumor pixel areas in the ultrasonic reference images is used to train a categorizer model with the coordination of a deep learning algorithm.

(e) Fifth step 15: An analysis of the ultrasonic image of the target liver tumor of the examinee is processed with the categorizer model to be provided to a clinician to determine the target liver tumor a liver tumor category and predict a risk probability of malignance of the target liver tumor. Thus, a novel method of IA for liver tumor is obtained.

The present invention uses an apparatus, comprising a SAT module 21 and an analysis module 22.

The SAT module 21 has an ultrasound probe 20.

The analysis module 22 connects to the SAT module 21 and comprises an image capturing unit 221, a reference storage unit 222, a control unit 223, a tumor marking unit 224, a classification unit 225, a comparison unit 226, and a report generating unit 227. Therein, the control unit 223 is a central processing unit (CPU) processing calculations, controls, operations, encoding, decoding, and driving commands with/to the image capturing unit 221, the reference storage unit 222, the tumor marking unit 224, the classification unit 225, the comparison unit 226, and the report generating unit 227.

For applications, the present invention is practiced in a computer. The control unit 222 is a CPU of the computer; the tumor marking unit 224, the classification unit 225, the comparison unit 226, and the report generating unit 227 are programs and stored in a hard disk or a memory of the computer; the image capturing unit 221 is a digital visual interface (DVI) of the computer; the reference storage unit 222 is a hard drive; and the computer further comprises a screen, a mouse, and a keyboard for related input and output operations. Or, the present invention can be implemented in a server.

On using the present invention, an ultrasound probe 20 of a SAT module 21 provides emission of SAT to an examinee from an external position corresponding to an area of liver to obtain an ultrasonic image of a target liver tumor of the examinee. During scanning, a doctor may perceive at least one ultrasound image of a suspected tumor to be selected as an ultrasonic image of a target liver tumor.

By using an image capturing unit 221, an analysis module 22 obtains the ultrasound image of the target liver tumor of the examinee, where the image is formed through imaging with the SAT module 21. A reference storage unit 222 stores a plurality of existing ultrasonic reference images of benignant and malignant liver tumors. A program is stored in an analysis module 22, where, on executing the program by a control unit 223, the program determines a liver tumor category to a clinician and predict a risk probability of malignance of the target liver tumor. The program comprises a tumor marking unit 224, a classification unit 225, a comparison unit 226, and a report generating unit 227.

The tumor marking unit 224 obtains coefficients and/or parameters coordinated with empirical data to automatically mark pixel tumor areas in the ultrasonic reference images and identify a plurality of liver tumor categories. For example, the tumor marking unit 224 may process marking based on physician experiences. The classification unit 225 obtains the pixel tumor areas in the ultrasonic reference images to process training by using a deep learning algorithm to build a categorizer model. The comparison unit 226 analyzes the ultrasonic image of the target liver tumor with the categorizer model to provide the clinician for determining the nature of the liver tumor of the examinee and further predicting a risk probability of malignance of the target liver tumor of the examinee. At last, the comparison unit 226 determines the liver tumor category and predicts the risk probability of malignance of the liver tumor by the clinician for the examinee to be inputted to the report generating unit 227 to produce a diagnostic report for assisting the physician in determining the nature of the liver tumor.

Thus, the present invention uses the abundant experiences of abdominal ultrasound specialists as a base to mark a pixel area of liver tumor in an ultrasound image. The parameters and coefficients of such empirical data are obtained for processing training by using the deep learning algorithm to establish the categorizer model having an accuracy ratio up to 86 percent (%). Hence, with the SAT image, a help to the doctor or ultrasound technician is immediately obtained through the present invention for determining the risk probability of malignance of the liver tumor and a base of reference is further provided for diagnosing the liver tumor category.

To sum up, the present invention is a method of IA for liver tumor, where SAT is coordinated with a deep learning algorithm to determine the risk probability of malignant liver tumor; by using coefficients and/or parameters coordinated with empirical data, pixel tumor areas in ultrasonic reference images are marked out to obtain a categorizer model having an accuracy up to 86% through the deep learning algorithm; and, thus, physicians are assisted with radiation-free and safe SAT to rapidly and accurately diagnose liver tumor categories.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of intelligent analysis (IA) for liver tumor, comprising steps of:
(a) first step: providing a device of scanning acoustic tomography (SAT) to scan an area of liver of an examinee from an external position to obtain an ultrasonic image of a target liver tumor of said examinee;
(b) second step: obtaining a plurality of existing ultrasonic reference images of benignant and malignant liver tumors;
(c) third step: obtaining a plurality of liver tumor categories from said existing ultrasonic reference images based on the shading and shadowing areas of said existing ultrasonic reference images to mark a plurality of tumor pixel areas in said existing ultrasonic reference images and identify said liver tumor categories of said tumor pixel areas;
(d) fourth step: obtaining said tumor pixel areas in said ultrasonic reference images to train a categorizer model with the coordination of a deep learning algorithm; and
(e) fifth step: processing an analysis of the ultrasonic image of said target liver tumor of said examinee with said categorizer model to provide said analysis to a clinician to determine said target liver tumor a liver tumor category and predict a risk probability of malignance of said target liver tumor.

2. The method according to claim 1,
wherein an analysis module and a SAT module connected to said analysis module are further obtained.

3. The method according to claim 2,
wherein said SAT module has an ultrasound probe to provide an emission of SAT to an examinee from an external position corresponding to an area of liver to obtain an ultrasonic image of a target liver tumor of said examinee.

4. The method according to claim 2,
wherein said analysis module comprises a control unit; an image capturing unit connected with said control unit; a reference storage unit connected with said control unit; a tumor marking unit connected with said control unit; a classification unit connected with said control unit; a comparison unit connected with said control unit; and a report generating unit connected with said control unit.

5. The method according to claim 4,
wherein said control unit is a central processing unit and processes calculations, controls, operations, encoding, decoding, and driving commands to said image capturing unit, said reference storage unit, said tumor marking unit, said classification unit, said comparison unit, and said report generating unit.

6. The method according to claim 4,
wherein said image capturing unit obtains an ultrasonic image of a target liver tumor of an examinee; and said image capturing unit is a digital visual interface (DVI).

7. The method according to claim 4,
wherein said reference storage unit stores a plurality of existing ultrasonic reference images of benignant and malignant liver tumors; and said reference storage unit is a hard drive.

8. The method according to claim 4,
wherein said tumor marking unit obtains a plurality of liver tumor categories from said existing ultrasonic reference images based on the shading and shadowing areas of said existing ultrasonic reference images to mark a plurality of tumor pixel areas in said existing ultrasonic reference images and identify said liver tumor categories of said tumor pixel areas.

9. The method according to claim 8,
wherein said tumor marking unit obtains coefficients and/or parameters coordinated with empirical data to automatically mark said pixel tumor areas appeared in said ultrasonic reference images.

10. The method according to claim 4,
wherein said classification unit obtains said tumor pixel areas in said ultrasonic reference images to train a categorizer model with the coordination of a deep learning algorithm.

11. The method according to claim 4,
wherein said comparison unit analyzes an ultrasonic image, which is of a target liver tumor of an examinee obtained by said image capturing unit, with a categorizer model, which is built by said classification unit, to provide a clinician to determine a liver tumor category and predict a risk probability of malignance of said target liver tumor of said examinee.

12. The method according to claim 4,
wherein said comparison unit provides said clinician to determine said liver tumor category and predict said risk probability of malignance of said liver tumor of said examinee to be inputted to said report generating unit to obtain a diagnosis report on the nature of said liver tumor.

13. The method according to claim 1,
wherein said liver tumor categories comprise benignant liver tumor categories and malignant liver tumor categories.
